(19) United States
(12) Patent Application Publication
(10) Pub. No.: US 2025/0352440 A1
(43) Pub. Date: Nov. 20, 2025

(54) COSMETIC PRODUCT IN THE FORM OF A FOAM

(71) Applicant: HENKEL AG & CO. KGAA, Düsseldorf (DE)

(72) Inventors: Thorsten KNAPPE, Schenefeld (DE); Alexandra GROSSDORF, Schenefeld (DE)

(21) Appl. No.: 18/871,429

(22) PCT Filed: Feb. 23, 2023
(86) PCT No.: PCT/EP2023/054564

§ 371 Date: Dec. 3, 2024

(30) Foreign Application Priority Data
Jun. 15, 2022 (DE) 10 2022 115 090.9

(51) Int. Cl. A61K 8/04; A61K 8/19; A61K 8/362; A61K 8/365; A61K 8/368; A61K 8/46; A61K 8/67; A61K 8/73; A61Q 19/00

(52) U.S. Cl. CPC A61K 8/046; A61K 8/19; A61K 8/362; A61K 8/365; A61K 8/368; A61K 8/463; A61K 8/676; A61K 8/735; A61Q 19/00

(57) ABSTRACT

A product is propelled as a foam or a spray from a container. A propellant gas is at least partly produced in situ in the container. A method for producing the product is also described.

The present invention relates to a product in a container, the product being used in the form of a foam or spray and the propellant gas required for this purpose being at least partly produced in situ in the container, and also to a method for producing the product according to the invention.

Products are provided in different forms in the household. The type of presentation is usually intended to allow improved handling or improved effect.

Cosmetic products are provided, for example, in the form of foams or sprays, in order to allow simple and uniform application (for example in hair care products or for hair styling). A uniform distribution or a pleasant feel are also important, for example in shower foams.

However, other products used in the home, such as washing and cleaning products for hard or soft surfaces, are also provided as a foam or spray. Oven cleaning sprays are intended to allow improved distribution. At the same time, the produced foam is intended to act better locally. The same applies to cleaning sprays or foams which are used in the bathroom for cleaning toilets or showers. Sprays, for example for pretreatment of stains, are also used in the cleaning of textiles, in order to allow local application.

However, sprays usually have to be pumped by hand, as a result of which the application is perceived as cumbersome. In the case of foams or aerosol sprays which are produced on the basis of a propellant gas, the question of sustainability is becoming increasingly important, since previous products mostly contain liquid gases as propellants, which are flammable and are known to be greenhouse gases, and which have a great impact on the carbon footprint. There is therefore a need for products used as a foam or spray which are more sustainable and as biodegradable as possible.

The liquid gases usually used to date can be replaced by inert gases such as nitrogen, carbon dioxide, or even compressed air. While nitrogen and compressed air are easy to handle in the production process, filling with carbon dioxide is more difficult. The advantage of carbon dioxide, however, is that it is partially soluble in an aqueous medium and, as a result, a) acts as a pressure accumulator in order to keep the pressure constant, and b) due to spontaneous outgassing and expansion of the gas when leaving the pressurized can, ensures better foaming in mousse applications or finer spray droplets when used in sprays.

The problem addressed by the present invention is therefore to provide products which form a fine foam or generate a fine spray mist. At the same time, the production is to be safe. In addition, the carbon footprint is to be as small as possible.

Surprisingly, it has been found that this problem is solved if the propellant, which is required to generate the foam or spray, is formed in situ in the interior of the container. In a first embodiment, the present invention thus relates to a product in a container which is used as a foam or spray, comprising at least one propellant precursor, CO₂ being released in situ from the propellant precursor in the container.

Surprisingly, it has been found that the known disadvantages in the use of CO₂ as the propellant are avoided by a propellant precursor. At the same time, CO₂ has a significantly lower carbon footprint than conventional propellant gases, in particular liquid gases, and is not flammable. The formation of foam or spray is excellent, and risks in the production of the products due to the propellant can be avoided.

According to the invention, the propellant CO₂ is produced only in the interior of the container. It is therefore not necessary to supply it as a gas or liquid, so that storage, transport and processing of CO₂ are omitted. Compared to products which are supplied directly with liquid CO₂, the production of products according to the invention is less technically complex and safer. Compared to products which are pressurized by means of liquid gas, the environmental impact is substantially smaller.

The product according to the invention is selected from cosmetic products, foodstuffs, detergents, spray paints, room fresheners or cleaning products. Cosmetic products, in the context of the present invention, are all products for cleaning and/or caring for and/or styling the human body, including the skin, hair, and nails. Washing products are, in particular, products which are used for washing and cleaning textiles, such as clothing, table linen, bath towels, etc. Room fresheners are usually perfume sprays for olfactory improvement of room air, and can contain further substances, for example for covering bad odors. Cleaning products are, in particular, products for cleaning hard surfaces, such as bath cleaners, all-purpose cleaners, glass cleaners, toilet cleaners, kitchen cleaners, hob and/or oven cleaners, etc. Foodstuffs are all products suitable for human consumption. In foamed form, they can either be consumed directly (spray cream, espuma, etc.) or used in the processing of foodstuffs (for example spray oil for spraying on pans, pots or baking molds). Spray paints are paints, provided in suitable containers, which can be applied uniformly to a surface by means of spray application.

The propellant precursor according to the invention is preferably solid or liquid (under normal pressure) and is thus simpler and safer to handle and dose than CO₂. CO₂ is released in the container in which the product is located. As a result, the pressure in the interior of the container increases slightly, as a result of which CO₂ is additionally dissolved in the liquid formulation.

The use according to the invention of a propellant precursor, by means of which CO₂ is produced as propellant in situ, also ensures particular properties of the product itself. In products which are used as foam, the foams have a reduced foam density compared to foams which are foamed exclusively by means of nitrogen or compressed air. An effect can also be seen in sprays or aerosol sprays (spray and aerosol spray are used synonymously in the present application). The droplet size is smaller in products according to the invention compared to sprays which are sprayed exclusively by means of nitrogen or compressed air. Both the lower foam density and the reduced droplet size result in the product according to the invention being able to be better distributed, thereby reducing the risk of local overdosing.

Suitable propellant precursors must be easy to handle, so that no additional risks occur during the preparation of the product according to the invention. In addition, the propellant precursor must be able to be dosed easily and exactly, in order to be able to adjust the amount of CO₂ produced. Furthermore, the propellant precursor is intended to be a cosmetically acceptable product which, when the product is used as a cosmetic product, causes no skin irritation or the like. The same applies in the event that the product according to the invention is a foodstuff. In this case, the corresponding legal requirements for foodstuffs are to be met.

Preferred propellant precursors are selected from metal carbonates or metal hydrogen carbonates or metal bicarbonates, the metal in particular being selected from alkali metals and/or alkaline earth metals. Examples include calcium carbonate, potassium carbonate, potassium bicarbonate, sodium carbonate, sodium bicarbonate, magnesium carbonate, etc. The propellant precursor is particularly preferably sodium bicarbonate or potassium bicarbonate, in particular sodium bicarbonate. These are simple to handle, can be obtained inexpensively, and can be combined well with conventional formulations for cosmetic products or washing and/or cleaning products. According to the invention, the product can have one, two or three or more different propellant precursors. The product according to the invention preferably has one or two propellant precursors. Particularly preferably, the product according to the invention has a propellant precursor.

It has been found that sodium bicarbonate is a particularly suitable propellant precursor. The propellant precursor therefore preferably comprises sodium bicarbonate (NaHCO₃) and in particular consists thereof. In a particularly preferred embodiment, the product according to the invention therefore comprises at least one active substance, and sodium bicarbonate (NaHCO₃) as the propellant precursor, CO₂ being released in the container in situ.

Depending on the exact composition of the product according to the invention, CO₂ can also be released in situ in the container due to the pH of the product itself. If this does not take place, the propellant precursor can, in preferred embodiments, be activated physically or chemically.

Physical activation can take place, for example, in that the product is exposed to heat before application. In this case, the temperature is dependent on the type of the product. Typical temperatures at which the propellant precursor is activated, and CO₂ is thus released in situ, are at 30° C. or more, in particular 40° C. or more, preferably 50° C. or more. Temperatures above 100° C., in particular above 120° C., and, above all, above 200° C., are less preferred, since an unnecessarily high amount of energy is then required for activation. The duration of the activation is a few seconds up to a few minutes, such as from 10 seconds to 30 minutes, in particular from 15 seconds to 20 minutes, particularly preferably from 30 seconds to 15 minutes. Within this period, a complete or approximately complete release of CO₂ usually takes place.

Alternatively and likewise preferably, it is possible for the propellant precursor to be chemically activated by at least one acid. In such a preferred embodiment, the product preferably further comprises at least one acid for releasing CO₂ from the propellant precursor. In a preferred embodiment, the present invention therefore relates to a product in a container which has at least one active substance and a propellant precursor, in particular sodium bicarbonate, and at least one acid, wherein CO₂ is released in situ from the propellant precursor in the container.

The product according to the invention can have one or more acids. The selection of the acid depends in particular on the type of product. A person skilled in the art can use acids which are conventionally used depending on the product. If the product is, for example, a cosmetic product, then the acid is preferably an acid approved for cosmetic use. Especially suitable are, for example, alpha hydroxy carboxylic acids, glycolic acid, salicylic acid, hyaluronic acid, ascorbic acid, ferulic acid, citric acid, lactic acid, etc. Lactic acid is particularly preferred, since it forms a lactate upon reaction with the propellant precursor, which lactate has nourishing properties.

If the product according to the invention is a washing or cleaning product, the acids already mentioned can also be used. In addition, mineral acids, such as hydrochloric acid, phosphoric acid, sulfuric acid or nitric acid, but also carbonic acid, acetic acid or hydrofluoric acid can also be used.

A product according to the invention can have one, two, three or more different acids. It preferably has one or two acids which react with the propellant precursor, so that CO₂ is produced in situ.

If the product according to the invention contains at least one acid, the amount of acid must be selected such that the propellant precursor is converted to CO₂ as completely as possible. The ratio is thus adapted to the type of the propellant precursor and the selected acid. One mole of protons from the acid should be present per mole of produced carbonate of the propellant precursor.

According to the invention, the amount of propellant precursor can be such that the amount of CO₂ produced is sufficient to set the pressure in the container such that it can be used by the end user.

According to the invention, it is also possible for the product to contain, in addition to the propellant precursor, at least one further propellant which is not CO₂. In this embodiment, the product which has the at least one active substance and the at least one propellant precursor is added to a container. The container is closed. It is then possible to increase the pressure in the container by adding a further propellant which is different to CO₂. In particular, nitrogen or air is used as the further propellant. The improved properties of the product resulting from the CO₂ produced in situ (lower foam density or smaller droplets) are retained even when a further propellant is used.

According to the invention, the product preferably has at least one active substance. This active substance is dependent on the type of the product, since, according to the invention, the product can be a cosmetic product, a room freshener, a spray paint, a foodstuff, a washing product, or a cleaning product. The active substance can be, for example, a surfactant. However, the active substance can also be a film-forming polymer, in particular if the product is a cosmetic product.

Cosmetic products, detergents and cleaning products are well known to a person skilled in the art. According to the invention, the propellant precursor can be added to conventional formulations. In particular, the propellant precursor can completely or partially replace liquid gas, nitrogen or compressed air in known formulations and improve product quality.

In a further embodiment, the present invention relates to a method for producing a product in a container, wherein the product is used as a foam or spray, comprising:
a) providing a container,
b) introducing at least one propellant precursor into the container,
c) introducing further components of the product into the container,
d) optionally introducing an acid into the container, and
e) closing the container.

Steps b), c) and d) according to the invention can be carried out in any order. The method according to the invention can furthermore comprise the step f)
f) increasing the pressure in the interior of the container with a propellant which is different to CO₂.

In a further embodiment, the present invention relates to the use of a product as described above as a foam or spray in use as a cosmetic product, foodstuff, room freshener, spray paint, detergent or cleaning product.

Features relating to preferred embodiments of the first embodiment, which are described above only in this regard, naturally also apply accordingly to further embodiments as features of preferred embodiments.

The following examples are intended to explain the subject of the present invention, without limiting it in any manner.

### EXAMPLES

The following products were produced:

| | Ex. 1 | Ex. 2 | Ex. 3 | Ex. 4 |
|---|---|---|---|---|
| | wt. % (active | wt. % (active | wt. % (active | wt. % (active |
| Ingredient | substance) | substance) | substance) | substance) |
| Active substances | 18 | 40 | 4 | 2 |
| Acid (e.g. lactic acid | 9.5 | 15 | 1 | 4 |
| 80%) | | | | |
| Sodium bicarbonate | 7.08 | 11.2 | 0.75 | 2.98 |
| Water (demineralized) | up to 100 | up to 100 | up to 100 | up to 100 |

The products (examples 1 to 4) were used once as a foam and as a spray. When used as an aerosol foam, nitrogen was used as a further propellant.

A foam was obtained which was described as pleasant and as easy to distribute by the subjects. The sprays exhibited a very fine spray pattern which could be applied uniformly to a surface.

1. A product configured to be propelled as a foam or spray from a container, the product comprising:
at least one propellant precursor,
wherein CO₂ is released in situ from the propellant precursor in the container.

2. The product of claim 1, wherein the at least one propellant precursor is selected from the group consisting of metal carbonates, metal hydrogen carbonates, metal bicarbonates, and mixtures thereof.

3. The product of claim 2, wherein the at least one propellant precursor is selected from the group consisting of calcium carbonate, potassium carbonate, potassium bicarbonate, sodium carbonate, sodium bicarbonate, and magnesium carbonate.

4. The product of claim 1, further comprising an acid for releasing the CO₂ from the propellant precursor.

5. The product of claim 4, wherein the acid is selected from the group consisting of alpha hydroxy carboxylic acids, glycolic acid, salicylic acid, hyaluronic acid, ascorbic acid, ferulic acid, citric acid, and lactic acid.

6. The product of claim 4, wherein the acid is selected from the group consisting of hydrochloric acid, phosphoric acid, sulfuric acid, nitric acid, carbonic acid, acetic acid, and hydrofluoric acid.

7. The product of claim 1, further comprising at least one further propellant different from CO₂.

8. The product of claim 1, wherein the product is a cosmetic product, a foodstuff, a detergent, a room freshener, spray paint, or a cleaning product.

9. A method for producing the product of claim 1, the method comprising:
introducing at least one propellant precursor into a container;
introducing at least one further component of the product into the container; and
closing the container.

10. The method of claim 9, further comprising:
increasing the pressure in the interior of the container with a propellant different from CO₂.

11. Use of the product of claim 1 as a cosmetic product, a foodstuff, a room freshener, spray paint, a detergent, or a cleaning product.

12. The product of claim 1, further comprising at least one active substance.

13. The product of claim 12, wherein the at least one active substance comprises a surfactant or a film-forming polymer.

14. The product of claim 2, wherein the metal is an alkali metal or an alkaline earth metal.

15. The product of claim 7, wherein the at least one further propellant comprises at least one of nitrogen or compressed air.

16. The method of claim 9, further comprising introducing an acid into the container before closing the container.

17. The method of claim 9, wherein the at least one further component comprises a surfactant or a film-forming polymer.

18. The product of claim 1,
further comprising an acid for releasing the CO₂ from the propellant precursor,
wherein the acid is selected from the group consisting of alpha hydroxy carboxylic acids, glycolic acid, salicylic acid, hyaluronic acid, ascorbic acid, ferulic acid, citric acid, lactic acid, hydrochloric acid, phosphoric acid, sulfuric acid, nitric acid, carbonic acid, acetic acid, and hydrofluoric acid, and
wherein the at least one propellant precursor is selected from the group consisting of metal hydrogen carbonates.

19. The product of claim 18,
wherein the acid is selected from the group consisting of alpha hydroxy carboxylic acids, glycolic acid, salicylic acid, hyaluronic acid, ascorbic acid, ferulic acid, citric acid, and lactic acid.
